(19) United States
(12) Patent Application Publication
(10) Pub. No.: US 2024/0082482 A1
(43) Pub. Date: Mar. 14, 2024

(54) MEMBRANE PLUNGER FLUID PRESSURE SWITCH

(71) Applicant: Becton, Dickinson and Company, Franklin Lakes, NJ (US)

(72) Inventors: Andrew J. BEAUPRE, Somerville, MA (US); Scott STEWART, Somerville, MA (US)

(73) Assignee: Becton, Dickinson and Company, Franklin Lakes, NJ (US)

(21) Appl. No.: 18/273,915

(22) PCT Filed: Jan. 27, 2022
(86) PCT No.: PCT/US2022/013996

§ 371 Date: Jul. 24, 2023

Related U.S. Application Data
(60) Provisional application No. 63/142,405, filed on Jan. 27, 2021.

(51) Int. Cl. A61M 5/142

(52) U.S. Cl. CPC A61M 5/14216

(57) ABSTRACT

A pressure switch is provided and includes an elastic membrane disposed such that pressure from fluid within a fluid path deforms the elastic membrane toward a plunger, thereby moving the plunger. The plunger is disposed between the membrane and an upper plate and is moveable along an axis extending between the membrane and the upper plate. A threshold pressure on the membrane from the fluid within the fluid path deforms the membrane, thereby moving the plunger toward the upper plate.

This application claims priority from U.S. Provisional Application 63/142,405, filed on Jan. 27, 2021 and PCTUS2022013996 filed Jan. 27, 2022, the entire contents of which is hereby incorporated by reference.

### BACKGROUND

### 1. Field

Apparatuses and methods consistent with example embodiments relate to a fluid pressure switch, and, more particularly, to a fluid pressure switch for use in an on-body medical device such as a wearable infusion pump.

### 2. Description of the Related Art

Diabetes is a group of diseases characterized by high levels of blood glucose resulting from the inability of diabetic patients to maintain proper levels of insulin production when required. Diabetes can be dangerous to the affected patient if it is not treated, and it can lead to serious health complications and premature death. However, such complications can be minimized by utilizing one or more treatment options to help control the diabetes and reduce the risk of complications.

The treatment options for diabetic patients include specialized diets, oral medications and/or insulin therapy. An effective method for insulin therapy and managing diabetes is infusion therapy or infusion pump therapy in which an insulin pump is used. An insulin delivery device (IDD) may include an insulin pump that can provide continuous infusion of insulin to a diabetic patient at varying rates in order to more closely match the functions and behavior of a properly operating pancreas of a non-diabetic person that produces the required insulin, and the insulin pump can help the diabetic patient maintain his/her blood glucose level within target ranges based on the diabetic patient's individual needs. Infusion pump therapy requires an infusion cannula, typically in the form of an infusion needle or a flexible catheter, that pierces the diabetic patient's skin and through which infusion of insulin takes place. Infusion pump therapy offers the advantages of continuous infusion of insulin, precision dosing, and programmable delivery schedules.

Anomalies or dysfunctions such as leaks, occlusions or presence of air bubbles in a fluid path can occur in an infusion pump and are not necessarily noticeable to the user. Detection of a dysfunction such as a partial or total occlusion along a fluid path in an infusion pump can be desirable to maintain accurately controlled medication delivery and to advise the user to discontinue use of a malfunctioning infusion device. A typical solution for occlusion detection is to place a pressure sensor in the infusion pump system and report occlusion when the pressure is above a certain threshold.

A pressure sensor, and all other portions of an IDD/insulin pump which come into contact with the fluid or fluid path therein must be subject to sterilization. However, sterilization and ageing can drastically change the elastomeric properties of membranes. Most fluid pressure switches rely on membrane deflection, or membrane switching, to measure pressure changes and have strict requirements for material characteristics and tight dimensional tolerances, particularly with respect to material thickness. However, the reliance on the membrane deflection, in conjunction with the ageing of the membrane and its exposure to sterilization cause problems with adherence to the strict requirements and precision of the switch.

### SUMMARY

Example embodiments may address at least the above problems and/or disadvantages and other disadvantages not described above. Also, example embodiments are not required to overcome the disadvantages described above, and may not overcome any of the problems described above.

According to an aspect of an example embodiment, a pressure switch is provided, the pressure switch comprising: an upper plate; a baseplate, opposite the upper plate, having an opening therein in communication with a fluid path; an elastic membrane sealing the opening in a baseplate, wherein the elastic membrane is deformable by pressure from fluid within the fluid path; a plunger disposed between the upper plate and the membrane and moveable along an axis extending between the upper plate and the membrane; and a spring, disposed between the upper plate and the plunger and exerting a pressure against the plunger. A threshold pressure on the elastic membrane from the fluid within the fluid path moves the plunger toward the upper plate.

The upper plate may comprise a printed circuit board; and the pressure switch may further comprise: at least one first conductive element disposed on an upper face of the plunger, and at least one second conductive element disposed on a lower face of the printed circuit board, such that the threshold pressure on the elastic membrane moves the plunger toward the upper plate such that the at least one first conductive element contacts the at least one second conductive element, closing an electrical circuit.

The pressure switch may further comprise: a compression standoff disposed between the upper plate and the baseplate and configured to maintain a predetermined spacing between the upper plate and the baseplate.

The at least one first conductive element may comprise a conductive layer disposed on the upper face of the plunger, and the at least one second conductive element may comprise a contact pad disposed on the lower face of the printed circuit board.

The spring may be metal or plastic.

The elastic membrane may comprise an elastomer.

According to an aspect of another example embodiment, an insulin delivery device is provided. The insulin delivery device comprises: a power and control system, and a pumping system controlled by the power and control system. The pumping system comprises: a fill port configured to receive a fluid medication from an external source, a reservoir configured to receive the fluid medication from the fill port and store the fluid medication therein, a cannula configured to deliver the fluid medication to a patient, a pump configured to move the fluid medication from the reservoir to the cannula, and a pressure switch, disposed on a fluid path between the fill port and the cannula. The pressure switch comprises: an upper plate; a baseplate, opposite the upper plate, having an opening therein in communication with the fluid path; an elastic membrane sealing the opening in a baseplate, wherein the elastic membrane is deformable by pressure from the fluid medication within the fluid path; a plunger disposed between the upper plate and the membrane and moveable along an axis extending between the upper plate and the membrane; and a spring, disposed between the upper plate and the plunger and exerting a pressure against the plunger. A threshold pressure on the elastic membrane from the fluid medication within the fluid path moves the plunger toward the upper plate.

The pressure switch may be located on the fluid path between the fill port and the reservoir.

The pressure switch may be located on the fluid path at the reservoir or can be built directly into the reservoir.

The pressure switch may be located on the fluid path between the pump and the cannula.

### BRIEF DESCRIPTION OF THE DRAWINGS

The above and/or other example aspects and advantages will become apparent and more readily appreciated from the following description of example embodiments, taken in conjunction with the accompanying drawings in which:

FIG. 1 is a cross section of a switch in a first position, according to an example embodiment;

FIG. 2 is a cross-section of the switch of FIG. 1 in a second position, according to an example embodiment; and

FIG. 3 is a block diagram of an IDD according to an example embodiment.

### DETAILED DESCRIPTION

Reference will now be made in detail to example embodiments which are illustrated in the accompanying drawings, wherein like reference numerals refer to like elements throughout. In this regard, the example embodiments may have different forms and may not be construed as being limited to the descriptions set forth herein.

It will be understood that the terms “include,” “including,” “comprise,” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,” “-er (-or),” and “module” described in the specification refer to an element for performing at least one function or operation, and may be implemented in hardware, software, or the combination of hardware and software.

Various terms are used to refer to particular system components. Different companies may refer to a component by different names—this document does not intend to distinguish between components that differ in name but not function.

Matters of these example embodiments that are obvious to those of ordinary skill in the technical field to which these example embodiments pertain may not be described here in detail.

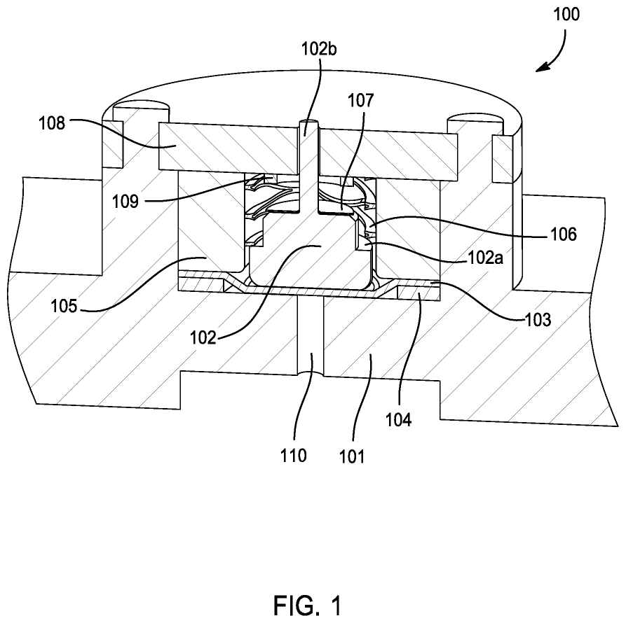

One or more example embodiments describe a pressure switch used to measure and trigger an alarm when the fluid contained in a flow path increases past a desired pressure point. FIG. 1 is a cross-sectional diagram of a fluid pressure switch in a first position, according to an example embodiment. The switch 100 includes a baseplate 101, a floating plunger 102, a hyper-elastic membrane 103, a gasket 104, a compression standoff 105, a spring element 106, a conductive layer 107, a printed circuit board (PCB) 108, and electrical contact pads 109. The switch 100 measures pressure of fluid in the flow path 110. The baseplate 101 may be part of a larger manifold.

The conductive layer 107 is disposed on the upper, flat surface of the plunger 102, and may either adhere to or be fused to this surface of the plunger 102.

The spring element 106 may be comprised of any of a number of materials including, but not limited to metal and plastic. Any of various types of springs may be used to achieve desired performance specifications including, but not limited to, compression springs, wave springs, and finger washers.

Two or more parts of the assembly of the switch may be combined to reduce complexity and assembly time. For example, two or more of the membrane 103, the gasket 104, and the compression standoff 105 may be a single, shot injection molded part.

Though the switch 100 is described as including two or more contact pads 109, other example, electrical hardware may be used in place of the contact pads, such as a small push button or light displacement sensor may be used to sense when the plunger 102 has reached the trigger point. Alternately, when the plunger 102 has reached the trigger point, a single electrical contact may be made with a single contact pad, with a second electrical contact being made with the spring itself.

Likewise another type of sensor, as would be understood by one of skill in the art, may be used to sense the plunger 102 reaching the trigger point.

The conductive layer 107 may be combined with the plunger 102 as a single element, or another means of completing the electrical circuit may be used in place of the conductive layer 107, such as conductive ink.

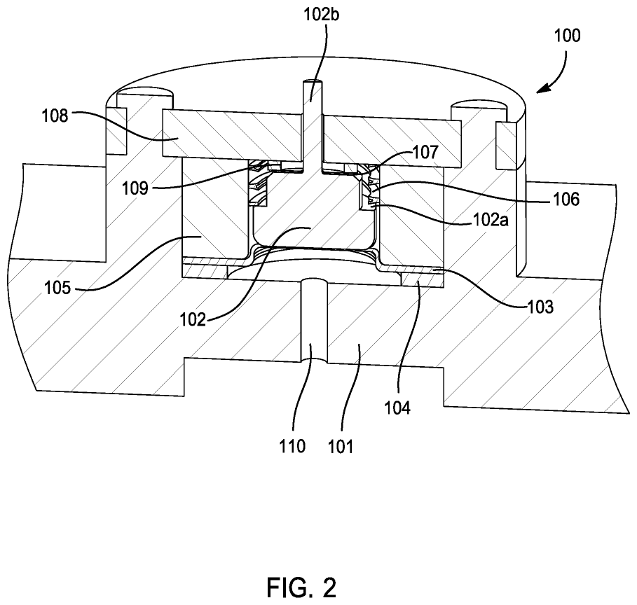

The example design, as shown in FIGS. 1 and 2 may be built up directly on the baseplate 101 or the components may be designed such that the switch can be pre-assembled before being attached to the baseplate 101. Alternatively, the configuration, as described may be flipped, such that the plunger is loaded into an alternate structure and pushed down toward the baseplate.

Alternately, the plunger 102 and the conductive layer 107 may be replaced with a conductive membrane, or the conductive layer 107 maybe disposed directly on the membrane 103, effectively eliminating the plunger 102.

The switch 100 uses the membrane 103 to transfer fluid pressure between an inlet of the fluid path 110 and the floating plunger 102. A counter force is applied to an outer radial ledge 102a of the floating plunder 102 by the spring element 106, which is held in a compressed state between the ledge 102a of the plunger 102 and a surface of the PCB 108. The PCB 108 may be heat staked with respect to the baseplate 101. A thru-hole 108a in the PCB 108 provide axial alignment and interfaces with a stem 102b extending from the plunder 102.

In addition to the spring 106, the PCB 108 also holds the compression standoff 105 which compresses both the membrane 103 and the gasket 104 against the baseplate 101. This compression forms a seal between the fluid path 110 and the plunger, thereby preventing any fluid from leaking from the fluid path 110 past the membrane 103. This seal maximizes the amount of pressure transferred from the fluid path 110 to the plunger 102. The compression standoff 105 also functions to constrain any non-axial motion of the plunger 102 by maintaining the plunger 102 within the confines of the space provided within the compression standoff 105, where the space may be cylindrical, as shown in FIGS. 1 and 2, but may alternately be another shape as would be understood by one of skill in the art.

FIG. 2 illustrates a cross-section of the switch 100 in a second position in which pressure in the fluid path 110 has increased pressure on the membrane toward the plunger, causing the plunger to move toward the PCB 108 against the pressure of the spring 106. As increasing fluid pressure is transferred through the membrane 103 to the plunger 102, the spring element 106 is compressed, and the plunger 102 advances toward the PCB 108. Once a trigger pressure is reached, the conductive layer 107 attached to the plunger 102 contacts the electrical contact pads 109 mounted on the membrane-facing side of the PCB 108, thereby completing a circuit. The switch trigger force is achieved when the force on the plunger 102 from the fluid pressure on the membrane 103 exceeds the counter force applied by the stretch of the elastic membrane 103 and the compression of the spring element 106.

Many related applications which use membrane deflection (or membrane stretching) to measure pressure changes have strict requirements for material characteristics and tight dimensional tolerances, particularly with respect to material thickness. According to this example embodiment, the switch 100 avoids the strict requirements by using a hyper-elastic membrane 103 which has a compliance that minimizes the membrane's contribution to the reaction force on the pressurized fluid and maximizes the contribution of the spring element. By shifting the majority of the reaction force on the fluid to the spring element, the trigger force of the switch 100 can easily be adjusted by changing a shape, size, compression amount, or material properties of the spring 106. This spring 106, which governs the pressure of the plunger 102 against the membrane 103 and the fluid, does not itself come into contact with the fluid. Also, the spring 106 may be a metal element or an elastic element.

According to this example embodiment, fluid is able to move up into the fluid path 110, and the fluid in the fluid path 110 applies a pressure against the membrane 103 and the plunger 102, so the plunger 102 moves toward the PCB 108 and the electrical contacts 109. A benefit of this example embodiment is that the switch 100 once activated by the contact of the conductive layer 107 with the contact pads 109, can be reset. The switch 100 may disengage when the pressure from the fluid decreases, and the plunger 102 and likewise the conductive layer 107 fall back to their resting position.

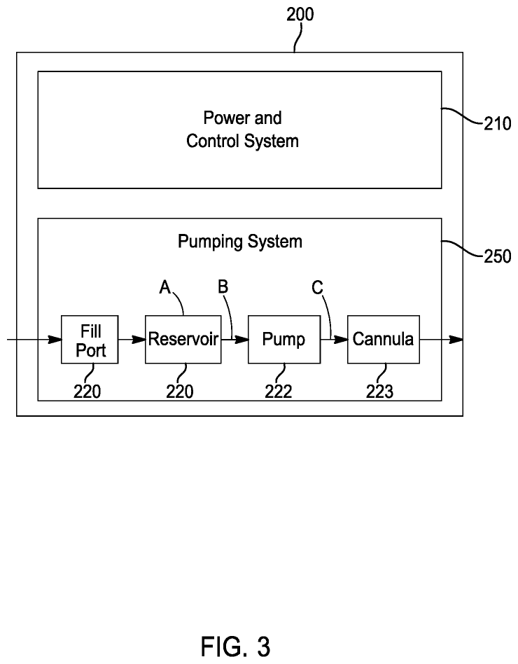

FIG. 3 is a block diagram of an IDD according to an example embodiment. It is to be understood that, although an example switch is described in conjunction with the example IDD as shown in FIG. 3, this is merely an example, and a switch in accordance with one or more example embodiments may be used in conjunction with any medication delivery system or medical device including a fluid path, as would be understood by one of skill in the art.

The IDD 200 is an example of a medical device configured for continuous subcutaneous delivery of insulin at set and variable basal (24-hour period) rates and bolus (on-demand) doses for the management of patients with type 2 diabetes mellitus requiring insulin therapy. The IDD 200 includes a power and control system 210, and a pumping system 250. The power and control system 210 may include one or more batteries for providing power for the IDD 200, a microcontroller, a memory, and additional electronics for control and regulation of the pumping system 250, as would be understood by one of skill in the art.

The pumping system 250 includes a reservoir 221 for storing a fluid medication (e.g. insulin) to be delivered, via a cannula 223, to a patient wearing the IDD 200. A pump 222 controllably delivers designated amounts of medication from the reservoir 221 through the cannula 223. The reservoir 221 may be filled via a septum or fill port 220 using a syringe. The IDD may also include a manual insertion mechanism (not shown) for inserting the cannula 223 into a patient.

A pressure switch according to an example embodiment described herein may be included within the pumping system 250 to detect pressure within a fluid path at any one or more locations between the fill port 220 and the cannula 223. For example, a switch 100 may be disposed at any one or more of location A, detecting a fluid pressure at the reservoir 221; location B, detecting a fluid pressure on a fluid path between the reservoir 221 and the pump 222; and location C, detecting a fluid pressure on a fluid path between the pump 222 and the cannula 223.

The pressure switch 100, disposed at one or more of the locations A, B, and C can aid in detection of an occlusion in the fluid path. Likewise, the pressure switch 100 can detect an overfill of the reservoir 221 and fluid path, providing an alert that the IDD is full. There is a need in medical devices such as an IDD to detect whether the medication is making its way into the patient. Accordingly, if there is an occlusion, the user needs to be alerted. Thus, there is a benefit to locating the pressure switch as far upstream in the fluid path as possible, as it would not be able to detect blockages upstream.

Many sources of friction may lead to variation in trigger pressures. This includes friction between the compression standoff 105 and the spring element 106, between the spring element 106 and the plunger 102, and between the plunger 102 and the compression standoff 105. These sources of friction may be mitigated by determination of the tolerances of the components to ensure that when the plunger 102 is at rest, it is already partially inserted in the compression standoff 105.

It may be understood that the example embodiments described herein may be considered in a descriptive sense only and not for purposes of limitation. Descriptions of features or aspects within each example embodiment may be considered as available for other similar features or aspects in other example embodiments.

While example embodiments have been described with reference to the figures, it will be understood by those of ordinary skill in the art that various changes in form and details may be made therein without departing from the spirit and scope as defined by the following claims.

What is claimed is:

1. A pressure switch comprising:
an upper plate;
a baseplate, opposite the upper plate, having an opening therein in communication with a fluid path;
an elastic membrane sealing the opening in a baseplate, wherein the elastic membrane is deformable by pressure from fluid within the fluid path;
a plunger disposed between the upper plate and the membrane and moveable along an axis extending between the upper plate and the membrane; and
a spring biasing the plunger toward the membrane;
wherein a threshold pressure on the elastic membrane from the fluid within the fluid path moves the plunger toward the upper plate.

2. The pressure switch according to claim 1, wherein:
the upper plate comprises a printed circuit board; and
the pressure switch further comprises:
at least one first conductive element disposed on an upper face of the plunger, and
at least one second conductive element disposed on a lower face of the printed circuit board, such that the threshold pressure on the elastic membrane moves the plunger toward the upper plate such that the at least one first conductive element contacts the at least one second conductive element, closing an electrical circuit.

3. The pressure switch according to claim 1, further comprising:
a compression standoff disposed between the upper plate and the baseplate and configured to maintain a predetermined spacing between the upper plate and the baseplate.

4. The pressure switch according to claim 3, wherein the compression standoff forms a seal between the fluid path and the plunger.

5. The pressure switch according to claim 3, wherein the compression standoff maintains the plunger within a space defined within the compression standoff.

6. The pressure switch according to claim 2, wherein the at least one first conductive element comprises a conductive layer disposed on the upper face of the plunger, and the at least one second conductive element comprises a contact pad disposed on the lower face of the printed circuit board.

7. The pressure switch according to claim 1, wherein the spring comprises one of metal and plastic.

8. The pressure switch according to claim 1, wherein the elastic membrane comprises an elastomer.

9. An insulin delivery device comprising:
a power and control system, and
a pumping system controlled by the power and control system, the pumping system comprising:
a fill port configured to receive a fluid medication from an external source,
a reservoir configured to receive the fluid medication from the fill port and store the fluid medication therein,
a cannula configured to deliver the fluid medication to a patient,
a pump configured to move the fluid medication from the reservoir to the cannula, and
a pressure switch, disposed on a fluid path between the fill port and the cannula, the pressure switch comprising:
an upper plate;
a baseplate, opposite the upper plate, having an opening therein in communication with the fluid path;
an elastic membrane sealing the opening in a baseplate, wherein the elastic membrane is deformable by pressure from the fluid medication within the fluid path;
a plunger disposed between the upper plate and the membrane and moveable along an axis extending between the upper plate and the membrane; and
a spring biasing the plunger toward the membrane;
wherein a threshold pressure on the elastic membrane from the fluid medication within the fluid path moves the plunger toward the upper plate.

10. The insulin delivery device according to claim 9, wherein:
the upper plate comprises a printed circuit board; and
the pressure switch further comprises:
at least one first conductive element disposed on an upper face of the plunger, and
at least one second conductive element disposed on a lower face of the printed circuit board, such that the threshold pressure on the elastic membrane moves the plunger toward the upper plate such that the at least one first conductive element contacts the at least one second conductive element, closing an electrical circuit.

11. The insulin delivery device according to claim 9, wherein the pressure switch further comprises:
a compression standoff disposed between the upper plate and the baseplate and configured to maintain a predetermined spacing between the upper plate and the baseplate.

12. The insulin delivery device according to claim 11, wherein the compression standoff forms a seal between the fluid path and the plunger.

13. The insulin delivery device according to claim 11, wherein the compression standoff maintains the plunger within a space defined within the compression standoff.

14. The insulin delivery device according to claim 10, wherein the at least one first conductive element comprises a conductive layer disposed on the upper face of the plunger, and the at least one second conductive element comprises a contact pad disposed on the lower face of the printed circuit board.

15. The insulin delivery device according to claim 9, wherein the spring comprises one of metal and plastic.

16. The insulin delivery device according to claim 9, wherein the elastic membrane comprises an elastomer.

17. The insulin delivery device according to claim 9, wherein the pressure switch is located on the fluid path between the fill port and the reservoir.

18. The insulin delivery device according to claim 9, wherein the pressure switch is located on the fluid path at the reservoir.

19. The insulin delivery device according to claim 9, wherein the pressure switch is located on the fluid path between the pump and the cannula.
